(19) United States
(12) Patent Application Publication
(10) Pub. No.: US 2009/0312294 A1
(43) Pub. Date: Dec. 17, 2009

(54) Method and Agent for Reducing Weight, Accelerating Lipid Catabolism, and/or Restricting Calories

(75) Inventor: Johannes Huber, Wien (AT)

Correspondence Address: LERNER GREENBERG STEMER LLP, P O BOX 2480, HOLLYWOOD, FL, 33022-2480, US

(73) Assignee: VALOR UNTERNEHMENSBERATUNG UND- BETEILIGUNG AG, Wien (AT)

(21) Appl. No.: 12/307,444

(22) PCT Filed: Jul. 5, 2007
(86) PCT No.: PCT/EP07/05936

§ 371 Date: Aug. 19, 2009

(30) Foreign Application Priority Data
Jul. 5, 2006 (EP) 06 450 093.7

(51) Int. Cl. A61K 31/56; A61P 3/04

(52) U.S. Cl. 514/171

(57) ABSTRACT

A method and an agent for reducing weight, accelerating lipid catabolism, and/or restricting calories includes a preparation having chrysin and cholic acid for producing a medicament.

The invention relates to an agent and method for reducing weight, accelerating lipid catabolism and restricting calories.

An excessive body weight in relation to body height is called overweight. In the narrower sense this means only what is termed pre-obesity, in contrast to serious overweight, obesity. The body mass index (BMI)—also the Kaup index—is an index for assessing human body weight. The body mass index is calculated as follows: BMI=mass (body weight in kilograms) divided by height (body height in meters) squared (BMI=M/H²). According to the WHO definition, overweight is a body weight corresponding to a BMI of 25 kg/m² and above, wherein a subdivision is performed into pre-obese (overweight in the narrower sense) (BMI=25-29.9), obese class I (BMI=30-34.9), obese class II (BMI=35-39.9) and obese class III (BMI>40).

Although it is unclear whether pre-obesity represents a health risk which is to be taken seriously (those who are slightly overweight (BMI=25-27) can in some circumstances have a higher life expectancy than those termed normal weight or highly overweight (BMI>27)), in the case of the truly obese (BMI of 30 and above) there is a significantly increased mortality risk. An important factor in the health impairment due to overweight, in particular in the risk of cardiovascular disorders, is also the distribution of the fatty tissue (“waist-hip ratio”).

Both overweight and increased fat consumption are associated with various cancer illnesses such as colon cancer or breast cancer, with metabolic syndrome, with the probability of infertility, diabetes, hypertension, arthrosis and heart disease. In addition, in the case of serious overweight, owing to the mechanical stress, damage to joints can occur, in particular in the knee.

Overweight can also be accompanied with psychosocial consequences: frequently, those affected feel excluded, or they exclude themselves socially. In addition to the actual pathological consequences of overweight, for most of those affected, overweight is, however, especially a cosmetic problem: overweight, owing to the prevailing modern idea of slimness, is considered unesthetic.

“Restricting calories” refers to limiting the energy consumed with the diet. Measures for restricting calories are frequently associated with an expectation of improved health and longer life. It has been found that restricting calories in humans has an action lowering cholesterol and blood pressure. On the basis of studies in various animals (primates, rats, mice, spiders, Drosophila, C. elegans, etc.), an action increasing lifespan is also ascribed to restricting calories. In the context of restricting calories, attempts are made to limit the calories supplied to the body to the minimum required, but nevertheless to ensure the supply of sufficient amounts of vitamins, minerals or other important nutrients. It is important in this case that the restriction in calories is not simply a mere food restriction, but is intended to provide a balanced diet. In this context, therefore use is frequently also made of the expressions “calorie restriction with optimal/adequate nutrition” (“CRON” or “CRAN”) or high-low diet (high in all nutrient components, low in calories).

The reason for the life-prolonging action of restricting calories is not clearly known. A plurality of explanations are currently being discussed, including the “hormesis hypothesis”, Sir2/SIRT-1 (“silent information regulator 2”)-based mechanisms, DHEA (dehydroepiandrosterone)-based mechanisms, the theory of free radicals, the theory of glycation, etc. Frequently, it is assumed that the changeover of cell metabolism to a reduced metabolic rate caused by calorie restriction leads to a reduced metabolic rate, as a result of which mitochondrial production of reactive oxygen species is decreased and the action of oxidative stress is thereby delayed.

Continuous calorie restriction requires a high degree of planning and discipline and is frequently combined only with difficulty with customary occupational and private habits and duties. Therefore, numerous proposals have been made for restricting calories by medicaments, by means of which the calorie restriction—with unrestricted dietary intake—can be achieved by a decrease or control of calorie uptake in the intestine. As an alternative to continuous calorie restriction, frequently, also, “interrupted fasts” are considered, in which, e.g., each second day, no food is consumed for 24 hours. Other alternatives relate to measures by operations (e.g. reduction in stomach size) or the use of biological substances or natural substances of which a similar action is known, for example resveratrol. Resveratrol is a phytoalexin which occurs in various plants and is marketed as food supplement.

Medicaments which lead to a reduction in calorie intake are being increasingly put on the market for preventing fat absorption (in addition to appetite suppressants or other slimming means). Examples of current medicament development are orlistat or sibutramine.

Orlistat is a synthetic derivative of lipstatin, a naturally occurring lipase inhibitor. Owing to the inhibition of lipase, decreased triglyceride cleavage occurs. As a result absorption of roughly one third of the fat taken up with the diet can be prevented. However, the administration of orlistat can lead to decreased absorption of the fat-soluble vitamins A, D, E and beta-carotene, owing to this mechanism of action.

Sibutramine leads to a central inhibition of serotonin reuptake and also to inhibition of reuptake of noradrenalin in the presynaptic neurones. Owing to the stimulating action on the sympathetic nervous system, however, sibutramine frequently also leads to an increase in blood pressure and heart rate.

It has been found that the agents and methods which are available in the prior art for weight reduction in general or for targeted fat catabolism or for calorie restriction in particular, are associated only either with a more or less high level of self discipline or else (by medicaments) with a not insignificant risk of side effects. There is therefore the need for a readily acceptable method for weight reduction, in particular for increasing (or for accelerating) fat catabolism, or for calorie restriction which can be employed without particular side effects and are based on natural metabolic mechanisms. Nevertheless, such a method must have satisfactory effectiveness and thus be able to be used in a simple manner, for example as food supplements.

Accordingly, the present invention relates to the use of a formulation comprising chrysin and cholic acid for producing a medicament for weight reduction, in particular for accelerating fat catabolism and for calorie restriction. With the combined formulation according to the invention an effective weight reduction, acceleration of fat catabolism and calorie reduction can be achieved. The combined administration of chrysin and cholic acid gives a significant synergistic effect which goes far beyond the effect which is known per se of chrysin on weight reduction (US 2004/0097429). The plant substances used according to the invention achieve in the combined formulation a synergistic action in that, firstly, increased displacement of stored energy proceeds, and secondly, reduction of food intake is ensured. The formulations according to the invention can be employed without particular side effects, with the components according to the invention being based on natural active ingredient principles and therefore on natural metabolic mechanisms.

Chrysin is a plant active ingredient which suppresses endogenous synthesis of estradiol. Since estrogen is an inducer of lipoprotein lipase which cleaves triglycerides into fatty acids and monoacyl glycerol and thereby enables the uptake of fatty acids in the adipocytes, the chrysin-related inhibition of lipoprotein lipase likewise reduces calorie uptake. Chrysin (5,7-dihydroxyflavone), a potent bioflavonoid, which occurs in honey, propolis and passionfruit inhibits primarily paracrine synthesis of estradiol from C19 steroids. The action on lipoprotein lipase thereby also falls, which leads to reduced fat deposition in the adipocytes. A chrysin-rich diet thereby inhibits fat incorporation. Chrysin also occurs in Pinea silvestris, this flavonoid was originally noticed owing to its androgenic partial activity. However, this is not so great that it would lead to hyperandrogenemic stress of the skin, hair, etc.

Cholic acid, which occurs, inter alia, in the leaves of pomegranate, causes firstly an improvement in insulin sensitivity (by occupying the PPaR-gamma receptor) and increased peripheral conversion of the endocrine-formed tetraiodothyronine into the biologically active triiodothyronine. As a result, increased calorie consumption is stimulated, which likewise leads to reduction in body weight. Whereas chrysin reduces the calorie intake, cholic acid stimulates calorie consumption. The energy balance is controlled by the thyroid hormone, wherein the thyroid itself forms the less active tetraiodothyronine which must be converted in the periphery into triiodothyronine. In the cell and/or the mitochondria, this effects the synthesis of anticoupling proteins and is involved, furthermore, in the control of ATP formation in the mitochondria. The conversion of tetraiodothyronine into triiodothyronine proceeds in the periphery via the enzyme deiodase which eliminates one iodine atom from the hormone formed in the thyroid gland and as a result first activates the compound. Deiodase is stimulated by cholic acid, as a result of which the body after food intake can keep its weight more or less stable. If more food is consumed, especially high-calorie fatty foods, more cholic acid is formed in the liver, as a result of which increased activation of deiodinase occurs in the periphery, and thereby increased conversion rate. Deiodinase is of variable activity and decreases with increasing age, as a result of which a relative triiodothyronine insufficiency occasionally occurs in the periphery.

As a result of the triiodothyronine activation according to the invention by cholic acid, the calories which are consumed are converted into energy via the uncoupling proteins and are not stored in adipocytes. As a result lipolysis is increased and body weight reduced.

Accordingly, the combination according to the invention of chrysin and cholic acid acts synergistically on reducing body weight, wherein chrysin inhibits the calorie uptake, whereas cholic acid increases energy conversion and calorie consumption.

As a result of the synergistic mode of action of the two main components in the formulation according to the invention, an outstanding activity can be induced, such that the formulation according to the invention can be used in a simple manner as food supplement and can be taken in isolation (for instance in unit dose form, e.g. as daily dose) or in combination with foods.

Preferably, the formulations according to the invention contain additionally isoflavones also. Isoflavones in this case support the action of chrysin, but from a different physiological approach. Isoflavones are glucosidase inhibitors which reduce the uptake of glycogen. Isoflavones are estrogen-like plant substances which preferentially occupy the beta estrogen receptor, but also independently thereof fulfill numerous functions in the mammal. They are also able to inhibit (1→6)-glucosidase. They thereby reduce glycogen metabolism and simultaneously also calorie uptake. Liponeogenesis in adipocytes makes use of fatty acids which are taken up via the diet and are eliminated from triglycerides by lipoprotein lipase. This process is greatly stimulated by locally formed estradiol. The biological background of this association between paracrine-formed estradiol and lipoprotein lipase is in the fact that pregnancy and three months of lactation additionally require 140 000 calories which is achieved via fat deposits of the Area gluteo femoralis. In this process, the sex hormone estrogen requires lipoid incorporation. In this case 17-beta estradiol is not formed in the ovary, but can be synthesized even in the adipocyte itself, since the fat cell has a high aromatase activity. In this case positive feedback is formed: estradiol stimulates lipoprotein lipase, as a result the adipocyte enlarges, as a result of which its aromatase activity increases which again leads to an increased provision of estrogen.

Glucosidases are enzymes which hydrolytically cleave glucosides, wherein the equilibrium is on the side of cleavage. Many glucosidases are group-specific, their specificity is directed towards the nature of the glycosidally bound sugar and the type of glycosidic bonds.

Glycogen and starch are hydrolyzed in the digestive tract first by amylases which are secreted into the intestinal tract. Alpha-amylases in saliva and in pancreatic juice hydrolyze alpha-(1→4)-bonds of external glycogen and amylopectin branches, forming D-glucose, a small amount of maltose and also dextrins. Dextrin is not further hydrolyzed by alpha-amylase since it cannot attack the alpha-(1→6)-bonds at the branching points. The enzyme alpha-(1→6)-glucosidase is required for this. This enzyme can hydrolyze the bonds at the branches and thereby break down the sugar to the extent that it can again be attacked by alpha-amylase. By means of the joint action of alpha-amylase and alpha-(1→6)-glucosidase, glycogen and amylopectin can thereby be completely broken down to glucose and to small amounts of maltose.

If glucosidase is inhibited, a considerable amount of the glycogen cannot be processed further and must in part be excreted with feces undigested. In preventive diabetology, glucosidase inhibitors are a possibility of facilitating weight reduction and thereby improving diabetes.

According to the invention, therefore, weight reduction proceeds via calorie restriction preferably by inhibiting the food uptake and/or by induction of calorie consumption, in particular by both mechanisms.

The formulation according to the invention is preferably provided in unit dose form, in particular as daily dose. Unit dose forms such as tablets, capsules, granules, etc., in particular also as foods or food supplements, have proved to be particularly advantageous for oral administration. Therefore, particularly preferably, the formulation according to the invention is particularly preferably provided in the form of tablets or capsules resistant to gastric juices. It has proved to be particularly suitable according to the invention to provide the formulation as a formulation which can be taken orally.

The relative ratios of the two or three (when isoflavones are also present) main components in the formulation according to the invention can be set on the one hand to the respective nutritional habits, or else with regard to the effect playing the main role. On the basis of the above described mode of functioning of the present invention, a person skilled in the art can in this case primarily target the inhibition of calorie intake or increase of the calorie consumption or of fat catabolism. More preferably, in the formulation according to the invention, isoflavones, chrysin and cholic acid, are provided in the following relative ratios (the percentage figures are in each case based on the total mass of the formulation in dry form or the total dry matter (in the case of liquid formulations)) based on the total weight of the active ingredients in the formulation (preferably in a unit dose form):

cholic acid: 5 to 90%,
chrysin: 5 to 90%, and
isoflavones: 0 to 90%.

Particular preference is given to the following relative ratios:

cholic acid: 10 to 50%,
chrysin: 20 to 60%, and
isoflavones: 5 to 40%.

According to a further preferred embodiment, the following minimum ratios can be provided (also in each case independently of one another):

cholic acid: at least 25%, in particular at least 30%,
chrysin: at least 25%, in particular at least 35%, and
isoflavones: at least 15%, in particular at least 35%.

Alternatively, the above described preferred relative ratios can also relate to the % (mass) fraction of the total active substances.

The administration according to the invention can be carried out either using a combined formulation or else in the form of an individual formulation set, in which a chrysin formulation, a cholic acid formulation and if appropriate an isoflavone formulation are provided. A “chrysin formulation”, “cholic acid formulation” and “isoflavone formulation” is taken to mean respectively formulations in which more than 50% of the active substances chrysin, cholic acid or isoflavones, respectively, are present in the formulation.

The individual components and relative ratios of the formulations according to the invention can also be set to a defined activation or inhibition action (e.g. preferred deiodinase activation activities and/or estradiol synthesis inhibition activities; preferably also certain glucosidase inhibition activities).

The formulations according to the invention can comprise the main components as sole active substances, or else in combination with further active ingredients. If appropriate, the formulation according to the invention can contain further active ingredients for weight reduction, for accelerating fat catabolism, and for calorie restriction, particularly preferably further natural substances, in particular resveratrol.

Preferably, the formulations according to the invention are produced from purified individual component formulations. In this case extracts or purified formulations of chrysin and cholic acid and also, if appropriate, of the further components are provided and mixed with one another in a suitable manner. The substances can have been isolated not only from natural sources but also be produced synthetically or semi-synthetically. In each case, the formulations to be administered must obviously be provided and administered in a form suitable for administration to humans.

Particularly preferred doses in the end product are:

chrysin: 10 to 5000 mg, in particular 100 to 500 mg
cholic acid: 10 to 5000 mg, in particular 100 to 300 mg
isoflavones: 0 to 500 mg, in particular 40 to 100 mg.

Preferably, the formulation according to the invention (and/or the chrysin formulation in the kit) contains 1 mg to 10 g, preferably 5 mg to 5 g, in particular 10 mg to 1 g (more preferably 100 mg to 500 mg) of chrysin.

Cholic acid is provided in the formulation preferably in an amount of 1 mg to 10 g, preferably from 10 mg to 5 g, in particular 100 mg to 1 g (more preferably 100 mg to 300 mg).

The isoflavones are preferably provided in the formulation according to the invention as a mixture of isoflavones, in particular as isoflavone extract from plant raw materials. Plant isoflavone extracts have proved themselves particularly suitable in this case. These are widely known in the prior art and are used as such for various purposes. According to a preferred embodiment, the isoflavones used according to the invention are formononetin, daidzein, genistein or mixtures thereof. Preferably, the formulation according to the invention generally contains 1 mg to 10 g, preferably 5 mg to 5 g, in particular 10 mg to 1 g (more preferably 40 mg to 100 mg) of isoflavones.

Also in this case, the above described amounts, instead of the combined formulation, can be provided in the form of a formulation kit, preferably in the above described amounts of active ingredients (that is to say preferably 1 mg to 10 g doses; e.g. a 50-500 mg solution, capsule or tablet of chrysin together with a 50-500 mg solution, capsule or tablet of cholic acid). For example, the set according to the invention contains, independently of one another
a chrysin formulation having 1 mg to 10 g, preferably 5 mg to 5 g, in particular 100 mg to 500 mg, of chrysin
a cholic acid formulation having an amount from 1 mg to 10 g, preferably from 10 mg to 5 g, in particular 100 mg to 300 mg, of cholic acid, and, if appropriate,
an isoflavone formulation having 1 mg to 10 g, preferably 10 mg to 1 g, in particular 40 mg to 100 mg, of isoflavones.

Also, in the set according to the invention, the formulations are preferably formulations which can be taken orally.

According to a further aspect, the present invention also relates to the use of the formulations according to the invention as food supplements for supporting weight reduction and calorie restriction, and also for accelerating fat catabolism. In this case the food supplements according to the invention can be taken either isolated (e.g. as capsules or tablets, before, during or after eating) or else in combination with the food itself (that is directly in the food itself), e.g. as daily dose all at once or 2-4 times per day.

Accordingly, the present invention also relates to a food containing an added formulation according to the invention. “Added” in this case means that chrysin or cholic acid are not naturally present in the food according to the invention or else are present in a concentration which is at least one, preferably at least two, in particular at least three, powers of ten below the concentrations in the food according to the invention. If a food naturally already contains a (trace) content of cholic acid or chrysin, according to the invention, by added formulation according to the invention this content (in % by mass (that is to say e.g. mg/g of total weight)) is raised at least one, preferably at least two, in particular at least three powers of ten with respect to chrysin or cholic acid. This food can be provided in many ways, e.g. as ready-to-eat dish, spread, bar, burger, yogurt, as fruit juice drink, etc. The dosage can of course be set simply by any food specialist, e.g. on the basis of the abovementioned amounts for obtaining the effects according to the invention.

In addition, the present invention also relates to the combined formulations according to the invention themselves, in particular for the medicinal use. In this case, it can be mixed in a manner known per se with a pharmaceutically acceptable carrier or diluent and finished to provide a pharmaceutical formulation. The formulation according to the invention is readily acceptable and can generally be taken completely safely even over relatively long periods of time.

Finally, the present invention also relates to the combined formulations according to the invention themselves, preferably for cosmetic use, in particular in cosmetic weight reduction.

The present invention will be described in more detail on the basis of the examples hereinafter to which it is obviously not restricted.

### EXAMPLE

### Clinical Study on the Action of the Formulations According to the Invention for Weight Reduction

Eight women between 52 and 59 in age who were hormonally in postmenopause and despite an activity program and targeted nutrition observed no change in weight, took 100 mg of chrysin for 5 days. This dose is under the dosages of any formulations which are offered as food supplements.

After a further 5 days, the patients were advised, in order to excite the conversion of peripheral T4 and peripheral T3, to take additionally 100 mg of cholic acid as supplement.

Intake proceeded in each case at midday after lunch.

The patients did not change their lifestyle and recorded their body weight each morning.

Table 1 shows the weight fluctuations after 5 days of chrysin and after a further 5 days of chrysin and cholic acid.

| | | kg after 5 days of |
|---|---|---|
| | | 100 mg of chrysin and |
| kg starting | kg after 5 days of | 5 days of 100 mg of |
| weight | 100 mg of chrysin | cholic acid |
| 66 | 65 | 64.2 |
| 72.5 | 71 | 70.05 |
| 78 | 78 | 76.5 |
| 71.2 | 70.2 | 70 |
| 68.4 | 67 | 65.8 |
| 75 | 73.5 | 72.8 |
| 70.3 | 69.5 | 68 |
| 69.5 | 69.5 | 69.5 |

Of the 8 patients, five exhibited a reduction of body weight owing to the chrysin supply, in three no weight reduction could be observed.

After a further 5 days on which not only chrysin but also cholic acid was taken, 7 of the 8 patients exhibited significant weight reduction, not only compared with the starting weight but also compared with day 5 and thereby the sole intake of chrysin.

No side effects occurred.

The patients felt subjectively well and some also reported an increase in libido.

### Interpretation of the Data:

The aromatase inhibitory action of chrysin is known. Estradiol stimulates the endothelial lipoprotein lipase and is therefore involved in the incorporation of triglycerides into the adipocytes. The aromatase inhibitory action apparently also decreases the lipoprotein lipase activity, simultaneously the androgen-specific lipolytic effect on the adipocytes occurs.

Cholic acid stimulates deiodinase and therefore the conversion of tetraiodothyronine into triiodothyronine. As a result—especially postprandially—the metabolism of the foods is stimulated. This could be the explanation for the fact that two completely independent mechanisms occur and finally, however, mediate an action, that is the reduction of body weight. In this case this is—depending on physiology—not an additive effect, since this is two completely different mechanisms which, in combination, have an effect reducing body weight over these two ways.

1-20. (canceled)

21. A method for weight reduction, in particular for accelerating fat catabolism and/or for calorie restriction, the method comprising the following steps:
using a medicament produced by a formulation including chrysin and cholic acid.

22. The method according to claim 21, which further comprises carrying out the weight reduction, the acceleration of fat catabolism and/or the calorie restriction by inhibition of food uptake and/or by induction of calorie consumption.

23. The method according to claim 21, wherein the formulation additionally contains isoflavones, the isoflavones being available in the formulation preferably as a mixture of isoflavones, in particular as isoflavone extract from plant raw materials.

24. The method according to claim 21, which further comprises using the formulation in unit dose form, in particular as a daily dose.

25. The method according to claim 21, which further comprises providing the formulation in the form of tablets or capsules being resistant to gastric juice.

26. The method according to claim 23, which further comprises providing the chrysin, the cholic acid and, optionally, the isoflavones, in the following relative ratios based on a total weight of active ingredients in the formulation:
cholic acid: 5 to 90%;
chrysin: 5 to 90%; and
isoflavones: 0 to 90%.

27. The method according to claim 26, which further comprises providing the cholic acid, the chrysin and the isoflavones in the following relative ratios, based on the total weight of the active ingredients in the formulation:
cholic acid: 10 to 50%;
chrysin: 20 to 60%; and
isoflavones: 5 to 40%.

28. The method according to claim 21, which further comprises further components for restricting calories, preferably further natural substances, in particular resveratrol.

29. The method according to claim 21, which further comprises providing from 1 mg to 10 g, preferably from 5 mg to 5 g, in particular from 100 mg to 500 mg, of chrysin in the formulation.

30. The method according to claim 21, which further comprises taking the formulation orally.

31. The method according to claim 21, which further comprises formononetin, daidzein, genistein or mixtures thereof as isoflavones in the formulation.

32. The method according to claim 21, which further comprises providing an amount of from 1 mg to 10 g, preferably from 10 mg to 5 g, in particular from 100 mg to 300 mg, of cholic acid in the formulation.

33. The method according to claim 21, which further comprises providing from 1 mg to 10 g, preferably from 10 mg to 1 g, in particular from 40 mg to 100 mg, of isoflavones in the formulation.

34. A food supplement for supporting weight reduction or calorie restriction, and/or for accelerating fat catabolism, the food supplement comprising:
the formulation according to claim 21.

35. A food, comprising:
an added formulation according to claim 21.

36. A combined formulation, comprising:
chrysin, cholic acid and optionally isoflavones, in particular for medicinal use.

37. A set, comprising:
a chrysin formulation and a cholic acid formulation, preferably for use for weight reduction, in particular for accelerating fat catabolism and/or for calorie restriction.

38. The set according to claim 37, which further comprises an isoflavone formulation.

39. The set according to claim 38, which further comprises, independently of one another:
a chrysin formulation having 1 mg to 10 g, preferably 5 mg to 5 g, in particular 100 mg to 500 mg, of chrysin;
a cholic acid formulation having an amount of from 1 mg to 10 g, preferably from 10 mg to 5 g, in particular 100 mg to 300 mg, of cholic acid; and
an optional isoflavone formulation having 1 mg to 10 g, preferably 10 mg to 1 g, in particular 40 mg to 100 mg, of isoflavones.

40. The set according to claim 37, wherein the formulations are to be taken orally.
